(19) United States
(12) Patent Application Publication
(10) Pub. No.: US 2026/0041911 A1
(43) Pub. Date: Feb. 12, 2026

(54) LOW FREQUENCY ELECTRODE TREATMENT DEVICE

(71) Applicant: Nu Eyne Co., LTD, Seoul (KR)

(72) Inventors: Dohyoung Kim, Seoul (KR); Joowan Seo, Seoul (KR); Pyungkyu Kim, Seoul (KR); Seol Jeon, Bucheon-si (KR); Kyeongwon Cho, Seoul (KR); Jisu Won, Seoul (KR); Taewan Kim, Gwangmyeong-si (KR)

(21) Appl. No.: 18/986,734

(22) Filed: Dec. 19, 2024

(30) Foreign Application Priority Data
Aug. 12, 2024 (KR) 10-2024-0107753

(51) Int. Cl. A61N 1/36; A61N 1/04; A61N 5/06

(52) U.S. Cl. CPC A61N 1/36034; A61N 1/0408; A61N 1/0472; A61N 1/36025; A61N 5/0622; A61N 2005/0647; A61N 2005/0651

(57) ABSTRACT

Provided is a low frequency electrode treatment device, and particularly, a low frequency electrode treatment device capable of performing the eye treatment and the sleep treatment simultaneously. The low frequency electrode treatment device, includes a substrate housing arranged in a forehead area on a rear surface of face frame so that a substrate equipped with a control circuit capable of performing at least one of eye treatment and sleep treatment is inserted, a substrate part for generating and controlling treatment waveform for performing at least one of eye treatment and sleep treatment, and an electrode module part, which is detachably provided in a module type so as to be electrically connected to the control circuit of the substrate part and performs stimulation treatment by electrodes, wherein the electrodes come into direct contact with at least two areas of user's facial skin and receive treatment signals transmitted according to electrical connection.

### TECHNICAL FIELD

The present invention relates to a low frequency electrode treatment device, and more particularly to, a low frequency electrode treatment device capable of performing the eye treatment and the sleep treatment simultaneously.

### BACKGROUND ART

A prior art patent document JP 7,285,632 discloses a low-frequency stimulation device aimed at providing cosmetic effects or relieving muscle fatigue, which includes a stimulation unit for stimulating a skin surface of a user, and a stimulation assisting unit positioned on a side corresponding to the skin surface to be stimulated and enhancing stimulation sensation.

Referring to FIG. 3 of the prior patent document, two electrodes are arranged as one pair horizontally spaced apart from each other on both sides of the nose.

In the prior patent document described above, stimulation treatment is only for cosmetic effects or relieving muscle fatigue, and is not suitable for eye treatment or sleep treatment. Furthermore, it is not possible to perform eye treatment or sleep treatment by the electrode arrangement of the prior patent document, and a method of connecting electrode channels that can perform eye treatment or sleep treatment is not disclosed.

### PRIOR ART DOCUMENT

### Patent Document

[Patent Document 0001] JP 7,285,632

### DETAILED DESCRIPTION OF THE INVENTION

### Technical Problems

Therefore, the present invention was created to solve the problems described above, and its purpose is to provide a low frequency electrode treatment device which can perform eye treatment and sleep treatment simultaneously.

However, the purpose of the present invention are not limited to the purpose mentioned above, and other purposes not mentioned will be clearly understood by those skilled in the art from the description below.

### Problem Solving Means

The above-mentioned objects of the present invention can be achieved by providing a low frequency electrode treatment device, which includes a substrate housing arranged in a forehead area on a rear surface of face frame so that a substrate equipped with a control circuit capable of performing at least one of eye treatment and sleep treatment is inserted, a substrate part for generating and controlling treatment waveform for performing at least one of eye treatment and sleep treatment, and an electrode module part, which is detachably provided in a module type so as to be electrically connected to the control circuit of the substrate part and performs stimulation treatment by electrodes, wherein the electrodes come into direct contact with at least two areas of user's facial skin and receive treatment signals transmitted according to electrical connection.

In addition, the electrodes of the electrode module parts operate to perform eye stimulation treatment in four areas of the facial skin when first treatment waveform selected for the eye treatment is supplied, and operate to perform sleep stimulation treatment in two areas of the facial skin when second treatment waveform selected for the sleep treatment is supplied.

In addition, the substrate part and the electrode module parts are detachably coupled by magnets, and the selected treatment waveform is transmitted through pogo pins during electric connection.

In addition, the low frequency electrode treatment device further includes temple parts, which are provided to be worn over user's ears and have silicon parts protruding from end portion areas with air-filled silicone so as to secure the treatment device to user's head and provide a cushion-like feeling.

In addition, the low frequency electrode treatment device further includes a treatment signal control unit, wherein when at least one treatment method of the eye treatment or the sleep treatment is selected, the treatment signal control unit generates, controls and transmits corresponding treatment signal waveform to the electrode module parts, and performs control so that a channel linkage method of the electrodes to which the treatment signal is transmitted is changed according to the treatment method selection of the eye treatment or the sleep treatment.

In addition, the treatment signal control unit performs control so that when the eye treatment is selected, the treatment signal supplied to the electrodes arranged in the four areas is channel-linked up and down through the electrodes but not channel-linked left and right, and when the sleep treatment is selected, the treatment signal supplied to the electrodes arranged in the two areas under user's eyes is channel-linked left and right through the electrodes but not channel-linked up and down.

In addition, the treatment signal control unit controls wavelength of at least two LEDs included in the substrate part so as to emit wavelengths that are helpful for sleep, when the sleep treatment is selected.

In addition, the electrode module part includes first and second electrode module parts, which are arranged horizontally apart from each other at a first interval on the forehead area of a user, and third and fourth electrode module parts, which are arranged horizontally apart from each other at a second interval on an under-eye area of the user.

In addition, each of the plurality of electrode module parts includes an electrode part, which comes in direct contact with the user's facial skin and is equipped with the electrodes for directly electrically stimulating the user's facial skin according to the selected treatment waveform, an upper electrode frame for supporting the electrode part, a lower electrode frame, which is arranged at a certain distance from the upper electrode frame and has magnetic pogo pins to be detachably attached by magnets, and a close-fitting adjustment part, which is interposed between the upper electrode frame and the lower electrode frame and contracts to allow the electrodes to adhere to a user's face according to shape of the user's face.

In addition, the electrode part has a surface shape formed in a curved arc shape like a bow so as to ensure close adhesion to the facial skin, and the close-fitting adjustment part is adjustable in length differently or equally for each of the four electrode modules so as to ensure a close fit to user's face shape.

In addition, the upper electrode frame includes a first bending adjustment clamp part, which extends in the downward direction and has a first fitting space formed in a central area for fitting, and a second bending adjustment clamp part, which is arranged in the horizontal direction at a certain distance from the first bending adjustment clamp part and has a second fitting space formed in a central area for fitting, and the lower electrode frame includes a first bending adjustment support part, which extends in the upward direction and is fitted into the first fitting space, and a second bending adjustment support part, which is arranged in the horizontal direction at a certain distance from the first bending adjustment support part and is fitted into the second fitting space.

In addition, with the fitting, a first tilt deformation of the electrode module parts in the left and right directions is freely adjusted in bending or tilting up to resistance of the close-fitting adjustment part, so that the left and right deformation is freely performed according to the face shape and the electrodes adhere close to the facial skin, and a second tilt deformation of the electrode module parts in the up and down directions is adjusted in bending or tilting relatively less than the first tilt deformation, so that the electrode module parts are prevented from bending when sliding down.

### Effect of the Invention

According to the present invention as described above, there is an effect that allows the eye treatment and the sleep treatment to be performed simultaneously.

### BRIEF DESCRIPTION OF THE DRAWINGS

The following drawings accompanied to the description illustrate preferred embodiments of the present invention, and serve to further understand the technical idea of the present invention together with the detailed description of the invention, so the present invention should not be interpreted as being limited to matters described in such drawings.

FIG. 1 and FIG. 2 are views illustrating a general configuration of a low frequency stimulation treatment device according to an embodiment of the present invention,

FIG. 3 to FIG. 5 are views illustrating a general configuration of an electrode module part according to an embodiment of the present invention,

FIG. 6 is a view illustrating first and second electrode support parts according to an embodiment of the present invention,

FIG. 7 is a view illustrating a cross-section of an electrode module part according to an embodiment of the present invention,

FIG. 8 is a view illustrating first and second bending adjustment clamp part and first and second bending adjustment support part according to an embodiment of the present invention,

FIG. 9 to FIG. 11 are views illustrating, in order, an electrode module part according to an embodiment of the present invention in a non-tilted state, a left-tilted state, and a right-tilted state,

FIG. 12 is a view illustrating channel linkage directions of electrodes in an eye treatment mode according to an embodiment of the present invention,

FIG. 13 is a view illustrating channel linkage directions of electrodes in a sleep treatment mode according to an embodiment of the present invention, and

FIG. 14 is a view illustrating a low frequency stimulation treatment device according to an embodiment of the present invention, in which all four electrode module parts are attached.

### BEST MODE FOR CARRYING OUT THE INVENTION

Hereinafter, a preferred embodiment of the present invention will be described with reference to the accompanying drawings. In addition, the embodiment described below does not unduly limit the contents of the present invention described in the claims, and it cannot be said that the entire configuration described in the present embodiment is essential as a solution to the present invention. In addition, the description of prior art and matters that would be obvious to those skilled in the art and may be omitted, and the description of such omitted components (methods) and functions may be sufficiently referenced within the range that does not deviate from the technical idea of the present invention.

A low frequency stimulation treatment device (or low frequency electrode treatment device) according to an embodiment of the present invention is a device that can treat either eye treatment (macular degeneration) or sleep treatment by stimulating facial skin with low frequency signals through electrodes. In other words, it is a device that can perform eye stimulation treatment or sleep stimulation treatment by changing a channel connection method of electrodes through which treatment signals (or waveforms) and treatment signals are transmitted. In particular, a user can independently select and use an eye treatment stimulation selection mode for eye treatment and a sleep treatment stimulation selection mode for sleep treatment. As described below, the treatment channel connection method of the electrodes varies depending on each treatment. Hereinafter, the low frequency stimulation treatment device according to an embodiment of the present invention will be described in detail with reference to the accompanying drawings.

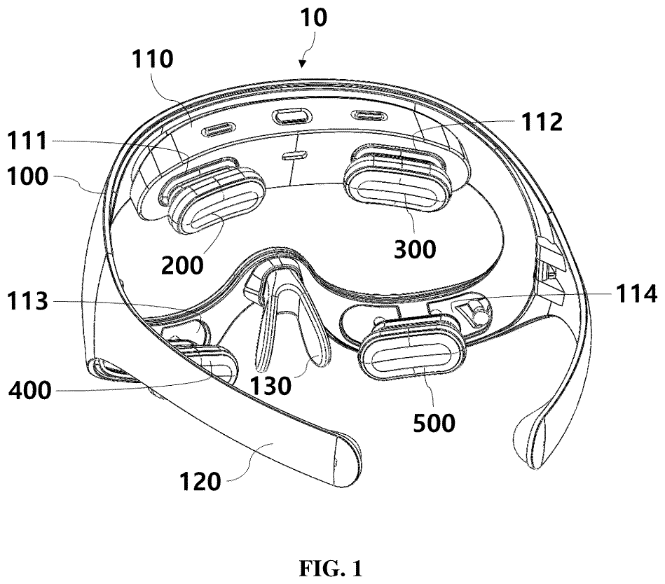

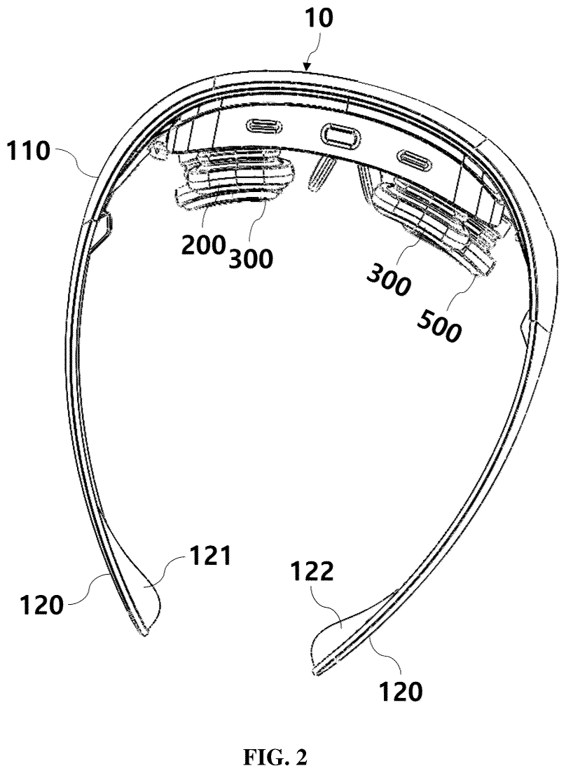

Referring to FIG. 1 and FIG. 2, a low frequency stimulation treatment device 10 according to an embodiment of the present invention generally includes a face frame 100, and first to fourth electrode module parts 200, . . . , 500. The face frame 100 is provided with a substrate housing 110, temple parts (120) and a nose pad part 130.

The substrate housing 110 is placed on a user's forehead area on a rear surface of the face frame 100. A substrate part (not shown) equipped with a control circuit capable of selectively performing eye treatment and sleep treatment is placed and fixed within the substrate housing 110.

The substrate housing 110 is provided with first, second, third and fourth electrode module insertion grooves 111, . . . , 114, into which a plurality of electrode module parts 200, . . . , 500 described later can be detachably attached by magnets, respectively. Each of the first, second, third and fourth electrode module insertion grooves 111, . . . , 114 is individually arranged with a magnet having sufficient magnetic force to be magnetically coupled to a magnetic pogo pin 251 described later.

The substrate part is provided with a power port that can be configured with various types of ports that can supply power from the outside, and may be provided with a rechargeable battery that can be charged by external power supply as desired according to control environment. In addition, the substrate part is also electrically connected to various physical buttons for operating the treatment device 10, such as buttons for turning the treatment device on or off or selecting a treatment mode.

The substrate part is provided with a treatment signal control unit, for example, an analog or digital circuit including a microcomputer, which generates and controls frequency waveform for eye treatment and sleep treatment. In addition, the treatment signal control unit controls and emits wavelengths of at least two sleep treatment LEDs provided on the substrate part when a sleep treatment selection mode is in operation.

In addition, the treatment signal control unit performs control so that when the eye treatment is selected, a treatment signal supplied to electrodes arranged in four areas is channel-linked up and down through the electrodes (up and down channel linking direction) but not channel-linked left and right, and when the sleep treatment is selected, a treatment signal supplied to electrodes arranged in two areas under user's eyes is channel-linked left and right through the electrodes (left and right channel linking direction) but not channel-linked up and down.

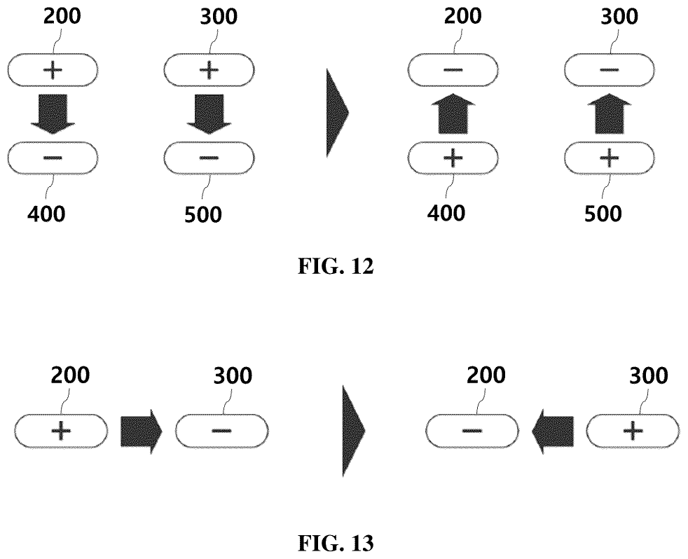

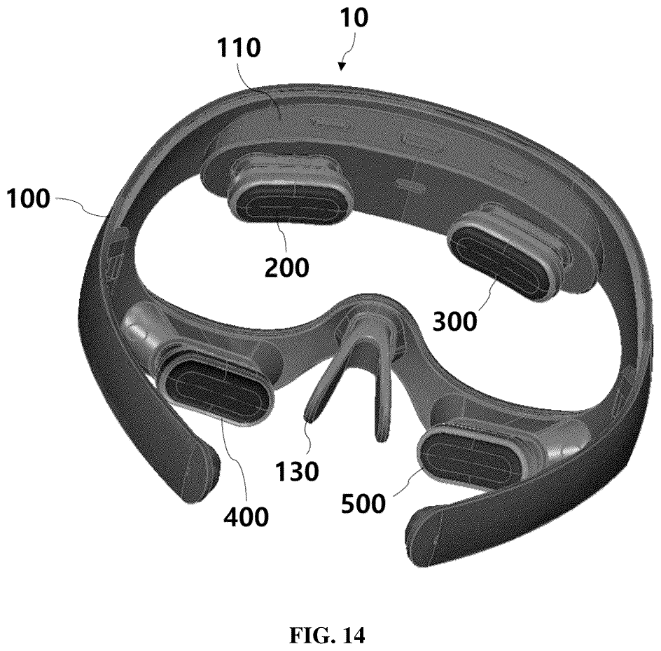

As an example, referring to FIG. 12, when the eye treatment is selected, the treatment signal control unit performs control so that the first electrode module part 200 and the third electrode module part 400 are mutually + and −channel-linked vertically through biphase signals, i.e., + and −signals, and similarly, the second electrode module part 300 and the fourth electrode module part 500 are mutually + and −channel-linked vertically.

Referring to FIG. 13, when the sleep treatment is selected, the treatment signal control unit performs control so that the first electrode module part 200 and the second electrode module part 300 positioned in a user's forehead area are mutually + and −channel-linked left and right through biphase signals, i.e., + and −signals, and the third electrode module part 400 and the fourth electrode module part 500 positioned under user's eyes do not operate.

As shown in FIG. 2, the temple parts 120 are worn over user's ears so as to secure the treatment device 10 to user's face. The temple parts 120 have silicon parts protruding from both end portion areas of the temple parts 120, with air-filled silicone so as to secure the treatment device 10 to user's head while providing a cushion-like feeling.

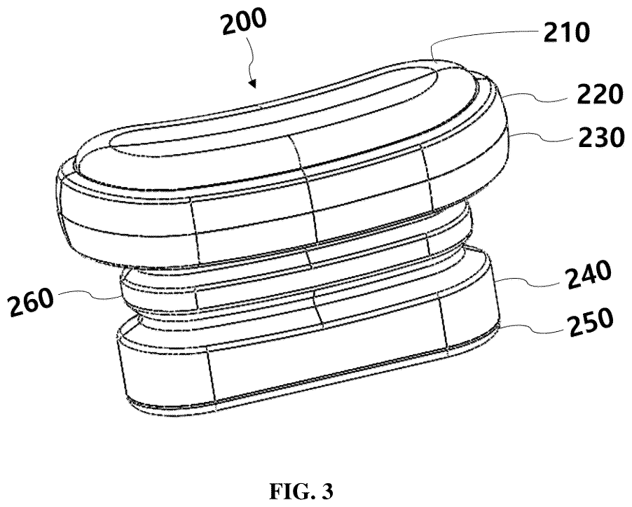

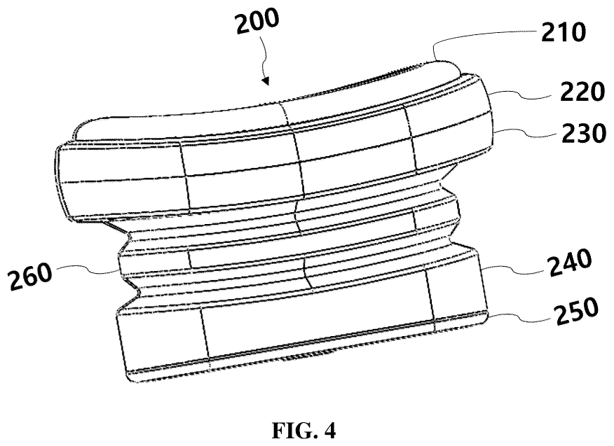

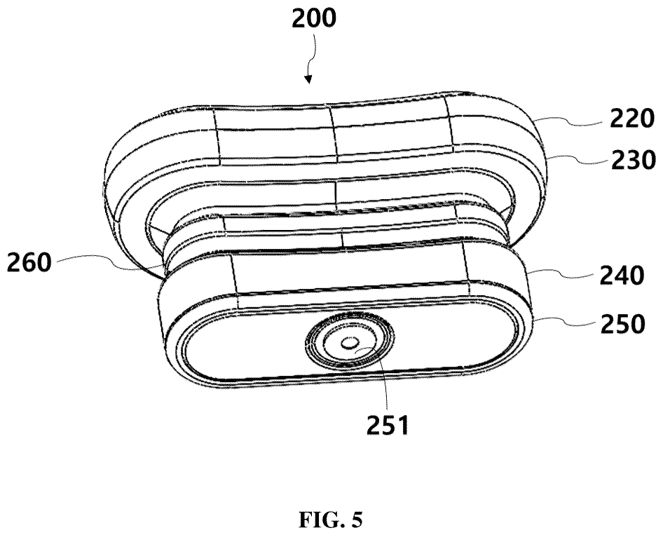

According to an embodiment of the present invention, a plurality of electrode module parts 200, . . . , 500 are detachably attached in a module type to the control circuit of the substrate part so as to be electrically connected to the control circuit of the substrate part, as shown in FIG. 3 to FIG. 5, and a treatment signal transmitted according to the electrical connection is transmitted to the electrodes, and the electrodes directly come into contact with at least two areas or four areas of user's facial skin, thereby performing stimulation treatment.

Each of the electrodes included in the plurality of electrode module parts 200, . . . , 500 operates in direct contact with the facial skin so as to perform eye stimulation treatment in the four areas of the facial skin when a first treatment waveform selected for the eye treatment is supplied, and operates in direct contact with the facial skin so as to perform the sleep stimulation treatment in the two areas of the facial skin when a second treatment waveform selected for the sleep treatment is supplied.

The substrate part and the plurality of electrode module parts 200, . . . , 500 described above are detachably coupled by the magnets, and when detachably attached by the magnets, electrical connection is made by the pogo pins so that the selected treatment signal is transmitted to the plurality of electrode module parts 200, . . . , 500 through the pogo pins.

The plurality of electrode module parts 200, . . . , 500 are composed of the first and second electrode module parts 200, 300, which are arranged horizontally apart from each other at a first interval on a forehead area of a user, and the third and fourth electrode module parts 400, 500, which are arranged horizontally apart from each other at a second interval on an under-eye area of the user. The first and second intervals may be equal to each other, or one may be shorter or longer than the other.

The configuration of each of the plurality of electrode module parts 200, . . . , 500 is identical to each other, so only the first electrode module part 200 will be described, and the description for the rest will be replaced by this description. However, a close-fitting adjustment part 260 (or bellows) provided to each of the plurality of electrode module parts 200, . . . , 500 may have different lengths so that close-fitting height can be adjusted to fit face shape.

The first electrode module part 200 includes an electrode part 210, an upper electrode frame 220, 230, a lower electrode frame 240, 250, and a close-fitting adjustment part 260, as shown in FIGS. 3 to 5.

The electrode part 210 is made of stainless steel and directly comes into contact with user's facial skin so as to directly electrically stimulate the facial skin according to a treatment signal generated in a selected treatment mode, wherein the electrode part 210 is formed in a fan-shaped ‘arc’ shape (or curved like a bow) so as to be closely fit to various face shapes of users. The electrode part 210 may protrude further than an electrode upper frame 220 of the upper electrode frame so as to come into directly contact with the skin.

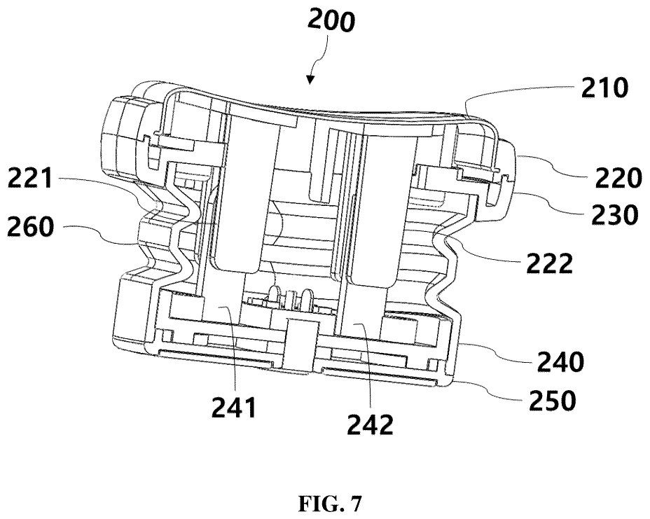

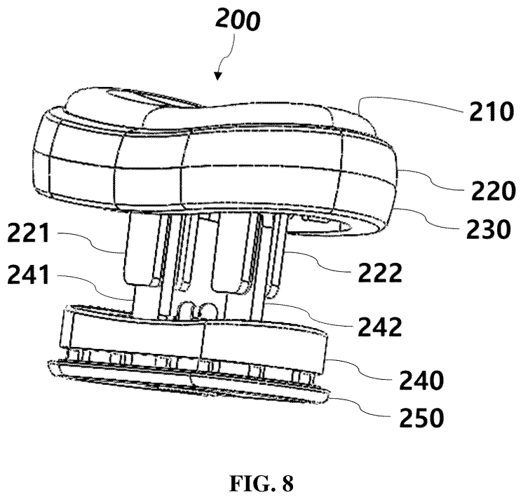

The upper electrode frame includes the electrode upper frame 220 and an electrode lower frame 230. The electrode upper frame 220 surrounds and supports the electrode part 210 in the circumferential direction. Referring to FIG. 7, a first bending adjustment clamp part 221 and a second bending adjustment clamp part 222 are arranged on an inner upper portion of the electrode upper frame 220 and protrude downwards. The first and second bending adjustment clamp parts 221, 222 are arranged at a certain distance apart from the inner portion of the electrode upper frame 220 and protrude downward so as not to touch a magnet upper frame 240 which will be described later.

The first bending adjustment clamp part 221 extends (protrudes) in the downward direction and has a first fitting space formed in a central area between both side tongs so that a first bending adjustment support part 241, which will be described later, can be fitted. That is, the first fitting space is formed in a space between first and second tongs that form the first bending adjustment clamp part 221.

The second bending adjustment clamp part 222 is arranged in the horizontal direction at a certain distance from the first bending adjustment clamp part 221, extends (protrudes) in the downward direction, and has a second fitting space formed in a central area between both side tongs so that a second bending adjustment support part 242, which will be described later, can be fitted. That is, the second fitting space is formed in a space between first and second tongs that form the second bending adjustment clamp part 222.

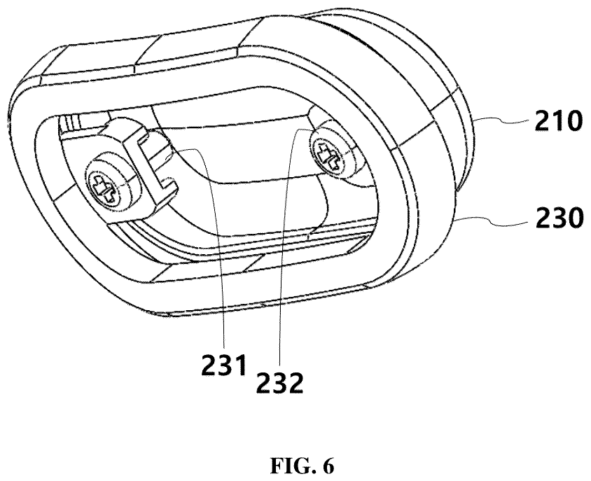

The electrode lower frame 230 is arranged on and coupled to a lower portion of the electrode upper frame 220. Referring to FIG. 6, first and second electrode support parts 231, 232 that can support the electrode part 210 are arranged inside the electrode lower frame 230. Each of the first and second electrode support parts 231, 232 includes a first support part, which protrudes in the central direction from an inner surface in a housing of the electrode lower frame 230, and a second support part, which is screw-coupled to the first support part and protrudes upward. The electrode lower frame 230 is coupled to one side of a close-fitting adjustment part 260, which will be described below.

The lower electrode frame includes the magnet upper frame 240 and a magnet lower frame 250. Referring to FIG. 7, the magnet upper frame 240 is coupled to one side of the close-fitting adjustment part 260. Therefore, the close-fitting adjustment part 260 is positioned in a space interposed between the electrode lower frame 230 and the magnet upper frame 240.

The first and second bending adjustment support parts 241, 242 are arranged on inside of the magnet upper frame 240 so as to protrude upward from a bottom surface of the magnet upper frame 240. An interval between the first and second bending adjustment support parts 241, 242 is approximately the same as the distance between the first and second bending adjustment clamp parts 221, 222 in the horizontal direction. Accordingly, the first bending adjustment support part 241 is fitted into the first fitting space, and the second bending adjustment support part 242 is fitted into the second fitting space.

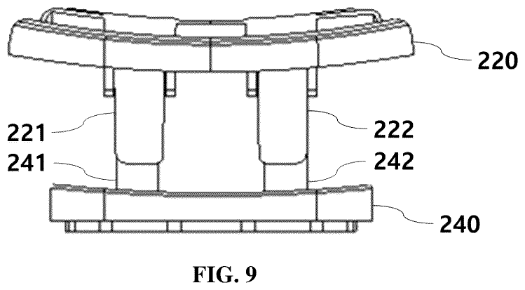

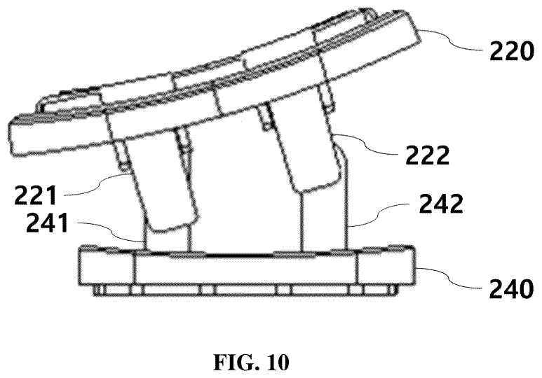

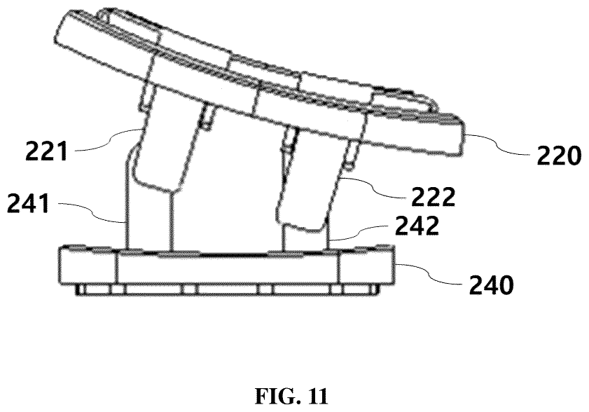

According to the fitting, first tilt deformation of the plurality of electrode module parts 200, . . . , 500 in the left and right directions (directions when the product is worn) is freely adjusted in bending or tilting up to resistance of the close-fitting adjustment part, so that the left and right deformation is freely performed according to the face shapes and the electrodes adhere close to the facial skin. That is, referring to FIG. 10, as an example, the first electrode module part 200 is tilted to the left with respect to FIG. 9 by axial rotation in the first and second fitting spaces, and referring to FIG. 11, the first electrode module part 200 is tilted to the right with respect to FIG. 9 by axial rotation in the first and second fitting spaces. Owing to the free deformation in the left and right directions, the electrodes can closely fit various face shapes, such as a person with a flat forehead and a person with a forehead that protrudes in the middle.

Second tilt deformation of the plurality of electrode module parts 200, . . . , 500 in the up and down directions (directions when the product is worn) is limited in bending or tilting adjustment by the first and second bending adjustment clamp parts 221, 222 and the first and second bending adjustment support parts 241, 242. That is, the second tilt deformation is adjusted in bending or tilting relatively less than the first tilt deformation, so that the plurality of electrode module parts 200, . . . , 500 are prevented from bending when sliding down. That is, the second tilt deformation is performed only for bending within a certain angle range.

The magnetic pogo pin 251 of each electrode module part is removably coupled with the magnet when inserted into each of the first to fourth electrode module insertion grooves 111, . . . , 114, wherein during the magnet coupling, a treatment signal is transmitted to each of the electrodes through the pogo pins.

Referring to FIG. 3, the close-fitting adjustment part 260 is interposed between the electrode lower frame 230 and the magnet upper frame 240. The close-fitting adjustment part 260 can be implemented with bellows because the close-fitting adjustment part 260 may contract when the electrodes are brought into close contact with a face according to the shape of a user's face.

It is advantageous that the electrode module parts can be fitted closely to a user's face to suit the user's face shape by adjusting the length of the bellows 260 provided in each electrode module part so that the length of each electrode module part can be made different overall or in pairs.

In explaining the present invention, explanations of prior art and matters obvious to those skilled in the art may be omitted, and explanations of such omitted components (methods) and functions may be sufficiently referred to within the scope that does not deviate from the technical idea of the present invention. In addition, the components of the present invention described above have been described only for the convenience of describing the present invention, and components not described herein may be added within the scope that does not deviate from the technical idea of the present invention.

The description of the composition and function of each part described above has been described separately for convenience of explanation, but if necessary, any one of the compositions and functions may be implemented by integrating it into another component or may be implemented in a more detailed manner.

Hereinabove, although the present invention has been described with reference to an embodiment, the present invention is not limited thereto, and various modifications and applications can be made. In other words, it would be readily apparent to those skilled in the art that many modifications can be made within a range that does not deviate from the gist of the present invention. In addition, it should be noted that if it is judged that a specific description of known features and functions related to the present invention or the connection relationship between each component of the present invention may unnecessarily obscure the gist of the present invention, the specific description has been omitted.

### Explanation of Reference Symbols

10: Low frequency stimulation treatment device
100: Face frame
110: Substrate housing
111: First electrode module insertion groove
112: Second electrode module insertion groove
113: Third electrode module insertion groove
114: Fourth electrode module insertion groove
120: Temple parts
121: First silicon part
122: Second silicon part
130: Nose pad part
200: First electrode module part
210: Electrode part
220: Electrode upper frame
221: First bending adjustment clamp part
222: Second bending adjustment clamp part
230: Electrode lower frame
231: First electrode support part
232: Second electrode support part
240: magnet upper frame
241: First bending adjustment support part
242: Second bending adjustment support part
250: magnet lower frame
251: magnetic pogo pin
260: Close-fitting adjustment part (or length adjustment part, bellows)
300: Second electrode module part
400: Third electrode module part
500: Fourth electrode module part

1. A low frequency electrode treatment device, comprising:
a substrate housing arranged in a forehead area on a rear surface of face frame so that a substrate equipped with a control circuit capable of performing at least one of eye treatment and sleep treatment is inserted;
a substrate part for generating and controlling treatment waveform for performing at least one of eye treatment and sleep treatment; and
an electrode module part, which is detachably provided in a module type so as to be electrically connected to the control circuit of the substrate part and performs stimulation treatment by electrodes, wherein the electrodes come into direct contact with at least two areas of user's facial skin and receive treatment signals transmitted according to electrical connection.

2. The device according to claim 1, wherein the electrodes of the electrode module parts operate to perform eye stimulation treatment in four areas of the facial skin when first treatment waveform selected for the eye treatment is supplied, and operate to perform sleep stimulation treatment in two areas of the facial skin when second treatment waveform selected for the sleep treatment is supplied.

3. The device according to claim 1, wherein the substrate part and the electrode module parts are detachably coupled by magnets, and the selected treatment waveform is transmitted through pogo pins during electric connection.

4. The device according to claim 1, further comprising temple parts, which are provided to be worn over user's ears and have silicon parts protruding from end portion areas with air-filled silicone so as to secure the treatment device to user's head and provide a cushion-like feeling.

5. The device according to claim 1, further comprising a treatment signal control unit, wherein when at least one treatment method of the eye treatment or the sleep treatment is selected, the treatment signal control unit generates, controls and transmits corresponding treatment signal waveform to the electrode module parts, and performs control so that a channel linkage method of the electrodes to which the treatment signal is transmitted is changed according to the treatment method selection of the eye treatment or the sleep treatment.

6. The device according to claim 5, wherein the treatment signal control unit performs control so that when the eye treatment is selected, the treatment signal supplied to the electrodes arranged in the four areas is channel-linked up and down through the electrodes but not channel-linked left and right, and when the sleep treatment is selected, the treatment signal supplied to the electrodes arranged in the two areas under user's eyes is channel-linked left and right through the electrodes but not channel-linked up and down.

7. The device according to claim 5, wherein the treatment signal control unit controls wavelength of at least two LEDs included in the substrate part so as to emit wavelengths that are helpful for sleep, when the sleep treatment is selected.

8. The device according to claim 1, wherein the electrode module part includes
first and second electrode module parts, which are arranged horizontally apart from each other at a first interval on the forehead area of a user, and
third and fourth electrode module parts, which are arranged horizontally apart from each other at a second interval on an under-eye area of the user.

9. The device according to claim 8, wherein each of the plurality of electrode module parts includes:
an electrode part, which comes in direct contact with the user's facial skin and is equipped with the electrodes for directly electrically stimulating the user's facial skin according to the selected treatment waveform;
an upper electrode frame for supporting the electrode part;
a lower electrode frame, which is arranged at a certain distance from the upper electrode frame and has magnetic pogo pins to be detachably attached by magnets; and
a close-fitting adjustment part, which is interposed between the upper electrode frame and the lower electrode frame and contracts to allow the electrodes to adhere to a user's face according to shape of the user's face.

10. The device according to claim 9, wherein the electrode part has a surface shape formed in a curved arc shape like a bow so as to ensure close adhesion to the facial skin, and the close-fitting adjustment part is adjustable in length differently or equally for each of the four electrode modules so as to ensure a close fit to user's face shape.

11. The device according to claim 9, wherein
the upper electrode frame includes:
a first bending adjustment clamp part, which extends in the downward direction and has a first fitting space formed in a central area for fitting; and
a second bending adjustment clamp part, which is arranged in the horizontal direction at a certain distance from the first bending adjustment clamp part and has a second fitting space formed in a central area for fitting, and
the lower electrode frame includes:
a first bending adjustment support part, which extends in the upward direction and is fitted into the first fitting space; and
a second bending adjustment support part, which is arranged in the horizontal direction at a certain distance from the first bending adjustment support part and is fitted into the second fitting space.

12. The device according to claim 11, wherein with the fitting, a first tilt deformation of the electrode module parts in the left and right directions is freely adjusted in bending or tilting up to resistance of the close-fitting adjustment part, so that the left and right deformation is freely performed according to the face shape and the electrodes adhere close to the facial skin, and a second tilt deformation of the electrode module parts in the up and down directions is adjusted in bending or tilting relatively less than the first tilt deformation, so that the electrode module parts are prevented from bending when sliding down.
